(19) United States
(12) Patent Application Publication
(10) Pub. No.: US 2023/0290515 A1
(43) Pub. Date: Sep. 14, 2023

(54) MACHINE LEARNING BASED PATIENT SPECIFIC POST-SURGERY MORTALITY PREDICTION SYSTEM AND RELATED METHODS

(71) Applicant: UNIVERSITY OF SOUTH FLORIDA, Tampa, FL (US)

(72) Inventors: Michael P. Rogers, Tampa, FL (US); Haroon Janjua, Tampa, FL (US); Paul C. Kuo, Tampa, FL (US); Abhinav Harish, Tampa, FL (US)

(21) Appl. No.: 18/174,471

(22) Filed: Feb. 24, 2023

Related U.S. Application Data
(60) Provisional application No. 63/314,343, filed on Feb. 25, 2022.
(60) Provisional application No. 63/482,202, filed on Jan. 30, 2023.

(51) Int. Cl. G16H 50/30; G06N 3/0442

(52) U.S. Cl. CPC G16H 50/30; G06N 3/0442

(57) ABSTRACT

Methods and systems for patient-specific post-surgery mortality prediction are disclosed. The methods and systems include: receiving a plurality of pre-operative factor indications for a patient; obtaining a first trained machine learning model and an interpretable model; applying the plurality of pre-operative factor indications to the first trained machine learning model to obtain a plurality of confidence values corresponding to the plurality of pre-operative factor indications; applying the plurality of confidence values to the interpretable model to obtain a plurality of interpretation indications, the plurality of interpretation indications corresponding to a subset of the plurality of pre-operative factor indications, the plurality of interpretation indications most contributing to mortality of the patient, the plurality of interpretation indications being specific to the patient; and outputting a survival probability of the patient based on the plurality of interpretation indications. Other aspects, embodiments, and features are also claimed and described.

### CROSS-REFERENCE TO RELATED APPLICATION(S)

This application claims the benefit of U.S. Provisional Patent Application Ser. Nos. 63/314,343 and 63/482,202, filed Feb. 25, 2022 and Jan. 30, 2023, respectively, the disclosures of which are hereby incorporated by reference in their entirety, including all figures, tables, and drawings.

### STATEMENT REGARDING FEDERALLY SPONSORED RESEARCH

N/A

### BACKGROUND

Operative decision making and patient counseling in surgery (e.g., high-risk cardiac surgery) can be nuanced and challenging because uncertainty of outcome may complicate the decision process to intervene. Risk prediction modeling has been implemented to better inform surgeons and patients and provide clinical decision support. However, these models rely on traditional statistical approaches and assume variables interact in a linear and cumulative fashion to impact patient outcome. However, evidence suggests the complex interplay of clinical variables on certain outcomes may not be adequately explained using traditional approaches and that variables may gain or lose impact significance due to the presence or absence of other features. What are needed is systems and methods that address one or more of these shortcomings.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects of the present disclosure, methods, systems, and apparatus for patient-specific post-surgery mortality prediction are disclosed. The methods and systems include: receiving a plurality of pre-operative factor indications for a patient; obtaining a first trained machine learning model and an interpretable model; applying the plurality of pre-operative factor indications to the first trained machine learning model to obtain a plurality of confidence values corresponding to the plurality of pre-operative factor indications; applying the plurality of confidence values to the interpretable model to obtain a plurality of interpretation indications, the plurality of interpretation indications corresponding to a subset of the plurality of pre-operative factor indications, the plurality of interpretation indications most contributing to mortality of the patient, the plurality of interpretation indications being specific to the patient; and outputting a survival probability of the patient based on the plurality of interpretation indications

In further aspects of the present disclosure, methods, systems, and apparatus for patient-specific post-surgery mortality prediction model training are disclosed. These methods, systems, and apparatus for patient-specific post-surgery mortality prediction model training may include steps or components for: receiving a plurality of training datasets corresponding to a plurality of patients, each of the plurality of training datasets comprising: a plurality of pre-operative factor indications; receiving a plurality of ground truth datasets corresponding the plurality of patients, each ground truth dataset comprising a subset of the plurality of pre-operative factor indications; and training a first machine learning model based on the plurality of training datasets and the plurality of ground truth datasets to obtain a plurality sets of confidence values, the plurality sets corresponding to the plurality of patients, the confidence values of each set of the plurality sets corresponding to a subset of the plurality of pre-operative factor indications, the subset most contributing to mortality of a patient corresponding to a respective set of the plurality sets.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a block diagram conceptually illustrating a system for patient-specific post-surgery mortality prediction according to some embodiments.

FIG. 2 is a flow diagram illustrating an example process for patient-specific post-surgery mortality prediction according to some embodiments.

FIG. 3 is a flow diagram illustrating an example process for model training for a patient-specific post-surgery mortality prediction model tool, according to some embodiments.

FIG. 4 illustrates receiver operator characteristic (ROC) curves for logistic regression and Gradient Boosting Machine (GBM) modeling according to some embodiments.

FIG. 5 illustrates example local interpretable model-agnostic explanations (LIME) modeling results individualized per patient according to some embodiments.

FIG. 6 illustrates an example approach to interpretable machine learning using a local interpretable model agnostic explanations (LIME) approach according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

In patients undergoing surgery (e.g., high-risk cardiac surgery), uncertainty of outcome may complicate the decision process to intervene. Some risk prediction modeling can calculate risk scores in a linear and cumulative fashion to impact patient outcome. However, evidence suggests the complex interplay of clinical variables on certain outcomes may not be adequately explained using traditional approaches and that variables may gain or lose impact significance due to the presence or absence of other features. The present disclosure provides patient-specific post-surgery mortality prediction systems and methods that can obtain patient specific mortality prediction (e.g., a post-surgery survival probability of the patient, contributing pre-operative factors, their corresponding contribution weights, any other suitable predictions). To provide patient specific mortality prediction, the systems and methods use at least two machine learning models (e.g., gradient boosting machine (GBM) modeling, local interpretable model-agnostic explanation (LIME) modeling, etc.) in a cascade manner. Thus, the systems and methods accurately predict mortality and identify the relative contribution of risk factors in individualized patients Thus, recognition and mitigation of existing risk factors that contribute to patient mortality prior to intervention may allow for improved patient outcome. Alternatively, the recognition of irreversible factors may assist in patient and family counseling when considering not proceeding with invasive procedures. In addition, utilization of this technique may uncover modifiable risk factors that allow for actionable intervention, resulting in a decreased risk of mortality.

### Example Patient-Specific Post-Surgery Mortality Prediction System

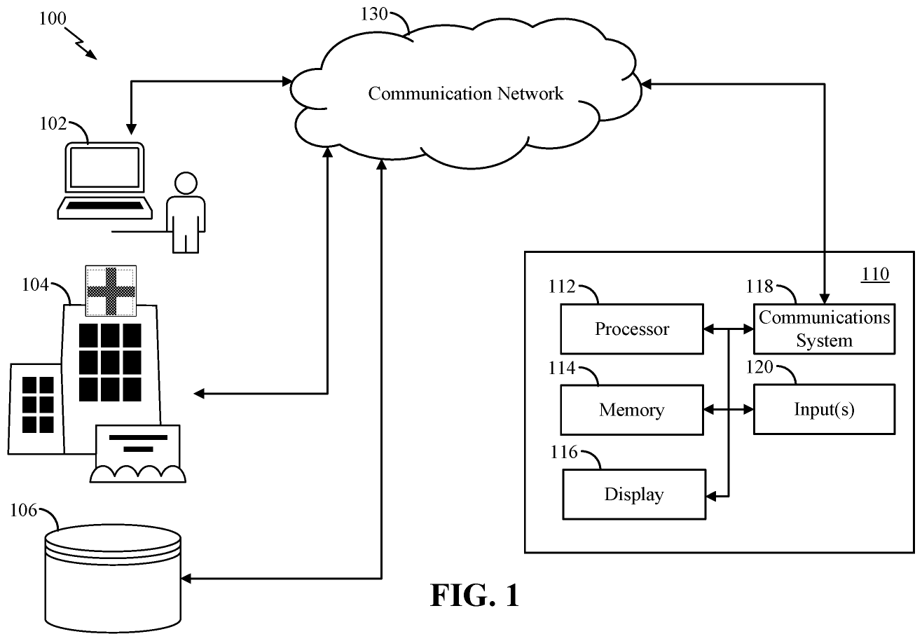

FIG. 1 shows a block diagram illustrating a system for patient-specific post-surgery mortality prediction according to some embodiments. As shown in FIG. 1, computing device 110 can receive multiple pre-operative factor indications (e.g., from a patient device 102, a facility, a clinic, a hospital, or any other suitable data source 106 about a patient), apply the multiple pre-operative factor indications to a first trained machine learning model, apply multiple confidence values from the first trained machine learning model to a second trained machine learning model to obtain multiple interpretation indications corresponding to a subset of the multiple pre-operative factor indications, and output a survival probability of the patient based on the multiple interpretation indications.

In some examples, computing device 110 can include processor 112. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc. Processor 112 may be located within a local (to the user) device (such as a mobile device), may be associated with a system hosting a patient medical record application, may be associated with a system providing information to physicians, may be part of a cloud-based resource, or otherwise, depending on the particular embodiment.

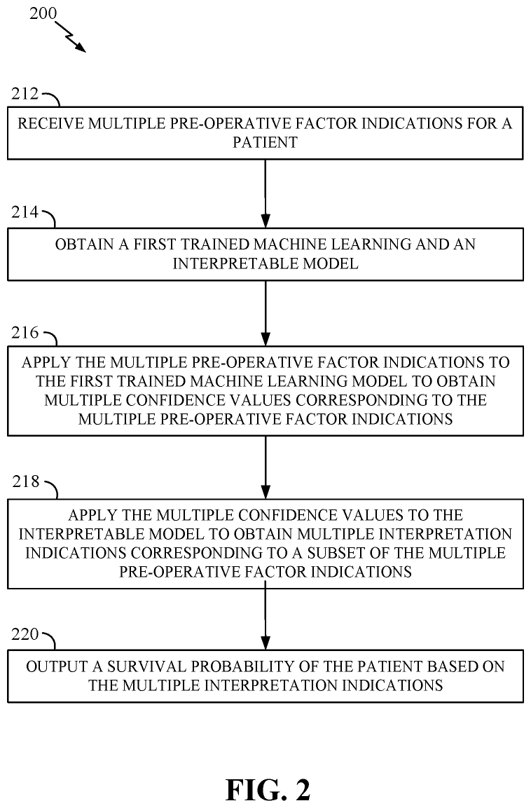

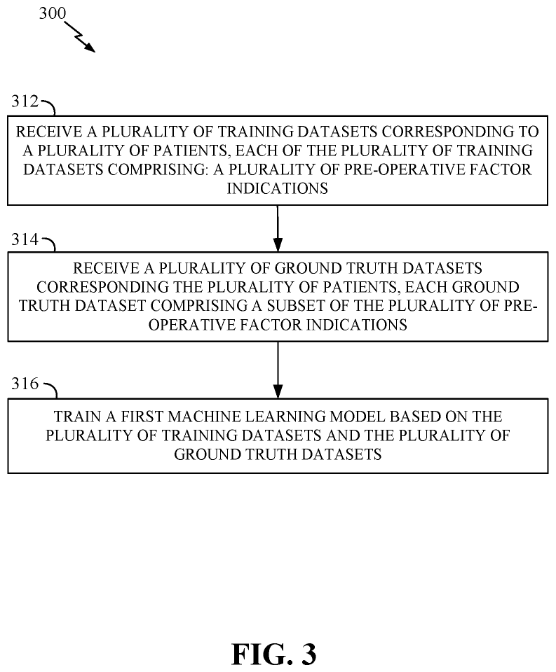

In further examples, computing device 110 can further include a memory 114. The memory 114 can include any suitable storage device or devices that can be used to store suitable data and instructions that can be used, for example, by the processor 112 to receive a plurality of pre-operative factor indications for a patient; obtain a first trained machine learning model and an interpretable model; apply the plurality of pre-operative factor indications to the first trained machine learning model to obtain a plurality of confidence values corresponding to the plurality of pre-operative factor indications; apply the plurality of confidence values to the interpretable model to obtain a plurality of interpretation indications; output a survival probability of the patient based on the plurality of interpretation indications; perform a combination of forward selection and a backward elimination to produce the plurality of pre-operative factor indications by reducing pre-operative factor dimensions; alter a first pre-operative factor indication of the plurality of pre-operative factor indications; monitor a resultant impact of the first pre-operative factor to the plurality of confidence values; and/or produce the multiple interpretation indications based on the resultant impact of the first pre-operative factor indication. The memory 114 can include any suitable volatile memory, non-volatile memory, storage, or any suitable combination thereof. For example, memory 114 can include random access memory (RAM), read-only memory (ROM), electronically-erasable programmable read-only memory (EEPROM), one or more flash drives, one or more hard disks, one or more solid state drives, one or more optical drives, cloud-based resources, etc. In some embodiments, the processor 112 can execute at least a portion of processes 200 and/or 300, described below in connection with FIG. 2 or 3.

In further examples, computing device 110 can further include communications system 118. Communications system 118 can include any suitable hardware, firmware, and/or software for communicating information over communication network 140 and/or any other suitable communication networks. For example, communications system 118 can include one or more transceivers, one or more communication chips and/or chip sets, etc. In a more particular example, communications system 118 can include hardware, firmware and/or software that can be used to establish a Wi-Fi connection, a Bluetooth connection, a cellular connection, an Ethernet connection, a local network, etc.

In further examples, computing device 110 can receive or transmit information (e.g., from or to a patient device 102, a facility, a clinic, a hospital 104, any other suitable data source 106, and/or any other suitable system) over a communication network 130. In some examples, the communication network 130 can be any suitable communication network or combination of communication networks. For example, the communication network 130 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 130 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, computing device 110 can further include a display 116 and/or one or more inputs 120. In some embodiments, the display 116 can include any suitable display devices, such as a computer monitor, a touchscreen, a television, an infotainment screen, etc. to display a report about patient-specific post-surgery mortality prediction, a survival probability of the patient, or any suitable information relating to the patient-specific post-surgery mortality prediction. In further embodiments, the input(s) 120 can include any suitable input devices and/or sensors that can be used to receive user input, such as a keyboard, a mouse, a touchscreen, a microphone, etc.

### Example Process

FIG. 2 is a flow diagram illustrating an example process 200 for patient-specific post-surgery mortality prediction in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., processor 112 with memory 114) in connection with FIG. 1 can be used to perform example process 200. However, it should be appreciated that any suitable apparatus or means for carrying out the operations or features described below may perform process 200.

At step 212, process 200 can receive multiple pre-operative factor indications for a patient. In some examples, multiple pre-operative factor indications can include at least one selected from the group of: patient co-morbidity related factor indications, laboratory test result indications, and patient demographics and disposition related factor indications. In some examples, the patient co-morbidity related factor indications can include at least one of: Diabetes mellitus with oral agents or insulin, Current smoker within one year, Dyspnea, History of severe COPD, Ascites, Congestive heart failure (CHF) in 30 days before surgery, Hypertension requiring medication, Acute renal failure, Currently on dialysis, Disseminated cancer, Open wound/wound infection, Steroid use for chronic condition, >10% loss body weight in last 6 months, Bleeding disorders, Transfusion>=1 units PRBCs in 72 hours before surgery, or Systemic sepsis. In some examples, the laboratory test result indications can include at least one of: Pre-operative WBC, Pre-operative hematocrit, Pre-operative platelet count, Pre-operative PTT, International Normalized Ratio (INR) of PT values, Pre-operative total bilirubin, Pre-operative SGOT, Pre-operative alkaline phosphatase, Pre-operative serum sodium, Pre-operative BUN, Pre-operative serum creatinine, or Pre-operative serum albumin. In some examples, the patient demographics and disposition related factor indications can include at least one of: Age Categories, Transfer Status, Patient Gender, Patient Race, Patient Ethnicity, Emergency case, Wound classification, Functional health status Prior to Surgery, BMI_Category, or Ventilator dependent. However, it should be appreciated that the patient co-morbidity related factor indications, the laboratory test result indications, and the patient demographics and disposition related factor indications are not limited to the lists described above and can include any other suitable indications. In some examples, process 200 can convert these indications in numeric form. For example, process 200 can ask the user if the user is “male or female” and this would be converted to 1 or 2 depending on the response, respectively.

In some examples, process 200 can receive the pre-operative factor indications for the patient from various sources (e.g., a patient device 102, a facility, a clinic, a hospital, a suitable database 106, or any other suitable data source for the patient). In some examples, the multiple pre-operative factor indications can be similar to data formats and values in the American College of Surgeons National Surgical Quality Improvement Program database. In other examples, the multiple pre-operative factor indications can have different data formats and values, which are suitable for training and using machine learning models.

In some examples, the multiple pre-operative factor indications can be a result of preprocessing. For example, process 200 can perform a combination of forward selection and a backward elimination to produce the multiple pre-operative factor indications by reducing pre-operative factor dimensions. In some examples, the multiple pre-operative factor indications (e.g., twelve or any other suitable pre-operative factor indications) relevant can be selected from pre-selected pre-operative factor indications (e.g., thirty-eight pre-operative factor indications, or any other suitable number of factor indications). In some examples, the pre-selected thirty-eight pre-operative factor indications can include multiple (e.g., six or any other suitable number) patient co-morbidity related factor indications, multiple (e.g., twelve or any other suitable number) laboratory test result indications, and/or multiple (e.g., ten or any other suitable number) patient demographics and disposition related factor indications. It should be appreciated that the pre-operative factor indications are not an exhaustive list. Any other suitable pre-operative factor indications can be added, and any recited pre-operative factor indication can be removed from the list. Among the preselected pre-operative factor indications, the multiple pre-operative factor indications can be selected by using a combination of backwards elimination (e.g., P>0.05 for exclusion) and forward selection (e.g., P<0.05 for inclusion) multivariate logistic regression modeling to exclude non-significant pre-operative factor indications or contributors to mortality. In some examples, some filtered pre-operative factor indications can be different from patient to patient. In other examples, some filtered pre-operative factor indications can be appliable to all patients.

In some examples, the forward selection is configured to select the most significant pre-operative factors, one after the other, until a predetermined threshold is reached. In further examples, the most significant pre-operative factor indications can be selected based on the smallest p-value, the highest increase in R², or any other suitable conditions. In further examples, the predetermined threshold can include a number of pre-operative factor indications or any other suitable threshold. For examples, the above-listed 38 pre-operative factors can be selected based on forward selection.

The backwards elimination is configured to remove the least significant pre-operative factors one after the other until another predetermined threshold is reached. In further examples, the least significant pre-operative factor indications can be selected based on the highest p-value, the lowest increase in R², or any other suitable conditions. In further examples, the predetermined threshold can include a number of pre-operative factor indications or any other suitable threshold. For example, among the above-listed 38 pre-operative factors or any suitable number of pre-operative factor indications, process 200 can remove 12 pre-operative factors and select 16 pre-operative factor indications or any suitable number of pre-operative factor indications. In some examples, the selected pre-operative factor indications can include at least one of: Diabetes mellitus with oral agents or insulin, Current smoker within one year, Dyspnea, History of severe COPD, Ascites, Congestive heart failure (CHF) in 30 days before surgery, Hypertension requiring medication, Acute renal failure, Currently on dialysis, Disseminated cancer, Open wound/wound infection, Steroid use for chronic condition, >10% loss body weight in last 6 months, Bleeding disorders, Transfusion>=1 units PRBCs in 72 hours before surgery, or Systemic sepsis. It should be appreciated that the selected pre-operative factor indications can be any other suitable pre-operative factor indications.

At step 214, process 200 can obtain a first trained machine learning model and an interpretable model. In some examples, the first trained machine learning model includes a gradient boost machine (GBM) model. It should be appreciated that it is not limited to the GBM model for the first trained machine learning model. For example, a fixed grid of Generalized Linear Models (GLM), a default Random Forest (DRF), five pre-specified GBMs, a near-default Deep Neural Net, an Extremely Randomized Forest (XRT), a random grid of XGBoost GBMs, a random grid of GBMs, a random grid of Deep Neural Nets, or multiple stacked ensemble models including combinations of these models can be employed. The first trained machine learning model can elucidate the relative contribution of individual pre-operative factors on mortality. In some examples, the interpretable model includes a local interpretable model-agnostic explanation (LIME) model. In some examples, the LIME model can produce the multiple interpretation indications by: altering a first pre-operative factor indication; monitoring a resultant impact of the first pre-operative factor indication to the plurality of confidence values; and producing multiple interpretation indications based on the resultant impact of the first pre-operative factor indication. In some examples, process 200 can alter more than one or all of pre-operative factor indications to produce the multiple interpretation indications. The LIME model can be performed on top of the trained GBM model and helps uncover details from GBM (or any other trained machine learning model) to draw individual insights from the model. In some examples, the first machine learning model produces beta coefficients, which are indicative of variable importance at the cohort level. The beta coefficients can be inputs to the LIME model to produce multiple interpretation indications which contribute most to the mortality of the patient.

As one example, the first machine learning model can be configured as a feedforward network, in which the connections between nodes do not form any loops in the network. As another example, a machine learning algorithm can be configured as a recurrent neural network (“RNN”), in which connections between nodes are configured to allow for previous outputs to be used as inputs while having one or more hidden states, which in some instances may be referred to as a memory of the RNN. RNNs are advantageous for processing time-series or sequential data. Examples of RNNs include long-short term memory (“LSTM”) networks, networks based on or using gated recurrent units (“GRUs”), or the like.

The first machine learning model can be structured with different connections between layers. In some instances, the layers are fully connected, in which each all of the inputs in one layer are connected to each of the outputs of the previous layer. Additionally or alternatively, neural networks can be structured with trimmed connectivity between some or all layers, such as by using skip connections, dropouts, or the like. In skip connections, the output from one layer jumps forward two or more layers in addition to, or in lieu of, being input to the next layer in the network. An example class of neural networks that implement skip connections are residual neural networks, such as ResNet. In a dropout layer, nodes are randomly dropped out (e.g., by not passing their output on to the next layer) according to a predetermined dropout rate. In some embodiments, a machine learning algorithm can be configured as a convolutional neural network (“CNN”), in which the network architecture includes one or more convolutional layers. In some embodiments, process 200 can use tensor flow lite to deploy the machine learning algorithm to a mobile device. In further embodiment, teachable machine can be used for training model. In further examples, a neural engine on the mobile device can perform the machine learning operation

At step 216, process 200 can apply the multiple pre-operative factor indications to the first trained machine learning model to obtain multiple confidence values corresponding to the multiple pre-operative factor indications. Each confidence value of a pre-operative factor indication can indicate a relative contribution level of the pre-operative factor indication on mortality of the patient. For example, sixteen pre-operative factor indications (e.g., labels and their values) are provided to the first trained machine learning model, the first trained machine learning model produces sixteen confidence values corresponding to the sixteen pre-operative factor indications to indicate relative contributions on mortality of the patient.

At step 218, process 200 can apply the multiple confidence values to the interpretable model to obtain multiple interpretation indications. In some examples, the multiple interpretation indications correspond to a subset of the multiple pre-operative factor indications. The subset of the multiple pre-operative factor indications can most contribute to mortality of the patient. The subset is specific to the patient. In some examples, a first interpretation indication of the multiple interpretation indications corresponding to a first pre-operative factor indication among the subset includes the first pre-operative factor and a weight of the first pre-operative factor, the weight being determined based on the resultant impact of the first pre-operative factor indication. The second trained machine learning model can produce interpretation indications corresponding to the subset of the multiple pre-operative factor indications. In some examples, the second trained machine learning model produces each of the subset of the plurality of pre-operative factor indications and a respective weight of each of the subset of the plurality of pre-operative factor indications on the survival probability of the patient. In some examples, the first machine learning model provides beta coefficients and variable importance at the cohort level. In further examples, the second trained machine learning model (e.g., a LIME model) provides explanations for individual patients rather than variable level information at the cohort level. Each individual will have varying degrees to which the variable will impact the user's or patient's mortality.

In some examples, the implementation of LIME is performed on top of the trained GBM model, process 200 can uncover details from GBM (or any other trained machine learning model) using LIME and can draw individual insights from the model. The final GBM model goes as input to LIME, and process 200 can specify the number of factors that need to be extracted for each patient from LIME. In some examples, process 300 is trained to produce top five contributing factors.

Furthermore, a standard machine learning model draws out prediction on a population level or for all the cases combined, whereas process 200 can draw actionable insights on individual patient level using LIME. Thus, using the second machine learning model, process 200 can produce individualized/personalized predictions. LIME works on each individual patient/record. Goodness of fit in terms of R-squared value can be generated for each prediction, and the positive or negative contribution of top factors can be easily visualized for effective decision making.

At step 220, process 200 can output a survival probability of the patient based on the plurality of interpretation indications. For example, the survival probability of the patient can be determined based on the sum of the multiple interpretation indications. In further examples, the survival probability of the patient can be determined based on the weighted sum of weights of the subset of the multiple pre-operative factor indications.

In some examples, process 200 can be used as an add-on in an electronic medical record system or as a standalone as a website (such as NSQIP, STS PROM score, etc.), where clinicians are able to go to input their patient's data and be provided with an individualized report of their patients factors and the degree to which they contribute to mortality. In some examples, process 200 can be used not only for pre-operative patient and family counseling but also as a way to identify modifiable risk factors significantly contributing to mortality to allow potential intervention.

In some embodiments, risk factors can be obtained from a patient's EMR upon request of the medical/surgical team considering a given surgical procedure. In some examples, information can be only displayed to the team for purposes of consideration, but is not displayed to the patient and does not become part of the EMR. In reverse, a hospital system could consider the high likelihood of a patient surviving, but the died, so the medical team can examine the procedure more carefully.

In further embodiments, healthcare insurers could use the survival probability and top contributing pre-operative factors or risk factors to approve/deny a given operation. When an insurance request is submitted, the system can calculate the survival probability and the top contributing pre-operative factors in response to the insurer's request. In some examples, the insurance company can reflect the mitigating factors to reduce the premium. In further examples, the insurance company could deny coverage until modifiable risk factors are adjusted by preventative care.

In further embodiments, the system deployed on a mobile device or as an add-on to an EMR. A patient enters factors or approves them being obtained from EMR. A probability and top contributing pre-operative factors can be output to the patient. In some examples, the system can update as patient changes risk factors. The system can provide recommendations for changing risk factors by running alternate scenarios and determining which factor is most likely to change outcome for the given patient. In further examples, the system could rank the easiest to achieve (e.g., weight loss).

### Example Process

FIG. 3 is a flow diagram illustrating an example process 300 for patient-specific post-surgery mortality prediction model training in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., processor 112 with memory 114) in connection with FIG. 1 can be used to perform example process 300. However, it should be appreciated that any suitable apparatus or means for carrying out the operations or features described below may perform process 300.

At step 312, process 300 can receive multiple training datasets corresponding to multiple patients. Each of the multiple training datasets can include: multiple pre-operative factor indications. In some examples, each training dataset is substantially similar to the multiple pre-operative factor indications at step 212 of FIG. 2.

At step 314, process 300 can receive multiple ground truth datasets corresponding the multiple patients. Each ground truth dataset can include a subset of the multiple pre-operative factor indications. In some examples, the high-risk CABG patients' data can be divided into training and testing cohorts. For examples, the train and test split were 70/30 percent respectively. The training data can further include the known outcome variable labels and test the predictive performance on the test data. The 70% training cohort was used to train the model, and to improve model training 10-fold cross validation technique can be used. The final set of variables from Logistic regression were used as input variables for GBM, and then GBM model was implemented.

At step 316, process 300 can train a first machine learning model based on the multiple training datasets and the multiple ground truth datasets to obtain multiple sets of confidence values. The multiple sets can correspond to the multiple patients. The interpretation indications of each set can correspond to a subset of the multiple pre-operative factor indications. The first machine learning model can include the GBM model.

### Example Data

Data Source and Patient Population: The inventors queried the American College of Surgeons National Surgical Quality Improvement Program database for adult patients undergoing coronary artery bypass grafting, open and transcatheter approaches to valve replacement, aortic root replacement, and valvuloplasty with a predicted 30-day post-operative mortality probability of 10% or greater as determined by the NSQIP calculator from 2012 to 2019. Cardiac surgical procedures represented in the Participant Use Data Files (PUF) were included for analysis. The NSQIP PUF contains patient-level aggregate data from participating academic and community hospitals in the United States and includes all major cases as determined by the Current Procedural Terminology (CPT®) code. Inter-Rater Reliability disagreement for selected participating hospitals is required to be less than 5% for database inclusion by NSQIP guidelines. Excluded cases followed standard NSQIP exclusion criteria and included patients under age 18, cases deemed “minor”, and those outside of cardiac surgical procedures of interest. This study was exempt from Institutional Review Board review and informed consent given the retrospective administrative database nature of this analysis. Reporting of results and predictive modeling followed recommended TRIPOD guidelines

Variable Selection and Predictive Modeling: Thirty-eight pre-operative variables included in the NSQIP risk calculator, including patient demographics, laboratory values, and pre-existing comorbid conditions were evaluated using a combination of backwards elimination (P>0.05 for exclusion) and forward selection (P<0.05 for inclusion) multivariate logistic regression modeling to exclude non-significant contributors to mortality. Data were split into training and testing cohorts of 70% and 30%, respectively, to train and -validate model performance on the testing cohort. Following variable selection, an automated machine learning approach with ten-fold cross validation consisting of three pre-specified XGBoost Gradient Boosting Machines (GBM) models, a fixed grid of Generalized Linear Models (GLM), a default Random Forest (DRF), five pre-specified GBMs, a near-default Deep Neural Net, an Extremely Randomized Forest (XRT), a random grid of XGBoost GBMs, a random grid of GBMs, and a random grid of Deep Neural Nets was employed. Multiple Stacked Ensemble models consisting of combinations of these models were trained throughout the process. Gradient boosting machine modeling was used for further investigation as this was the top performing model by area under the curve (AUC).

The resultant significant factors surviving regression were used in modeling to elucidate the relative contribution of individual factors on mortality. Ten-fold cross validation was used as a resampling technique to substantiate machine learning model performance. Diagnostic ability and model performance of logistic regression and GBM algorithms was evaluated by receiver operator characteristic (ROC) area under the curve.

The resultant features of GBM modeling were further explored using a local interpretable model-agnostic explanations (LIME) modeling approach. LIME is an analytical method to explain predictions of a regressor by approximating it locally with an interpretable model. Through the modification of a single data sample by altering the feature value and subsequently observating the resultant impact on outcome, the algorithm attempts to “interpret” predictions from each sample. The output provides a set of interpretations that represent the contribution of each feature to a prediction for each individual patient (i.e., local interpretability). Further, factor combinations of the top five contributors to mortality in the overall cohort were further investigated to explore the additive effect of these predictors using permutation confusion matrices in combinations of two or more variables. These combination matrices were evaluated by negative predictive value, accuracy, sensitivity, and specificity.

Data preparation, cleaning, and computation of descriptive statistics was performed using (e.g., Stata software version 16 (StataCorp, College Station, Tex.). Analysis was conducted in R (R Core Team, 2014) using R Studio (RStudio, PBC, Boston, Mass.)). Quantitative data are reported as number and percentage (n, %). Pearson's chi-square was used to compare baseline patient demographics and pre-operative risk factors. A P-value<0.05 was considered significant.

Results: A total of 1,291 cardiac surgeries corresponding with an ACS-NSQIP predicted mortality≥10% were included for analysis. There were 194 patient deaths (15.03% observed mortality) among thirty-four unique cardiac surgical procedures. Those contributing most to mortality included coronary artery bypass grafting with a single arterial graft, open mitral valve replacement, and ascending aortic replacement with valve resuspension. Univariate analysis between alive and deceased cohorts demonstrated deceased patients were more likely overweight or obese (38.66% vs 31.45%, P<0.05), to present in transfer from an outside emergency department (18.56% vs 11.03%, P<0.05), have cases designated American Society of Anesthesiology (ASA) Physical Status Classification Class V (20.1% vs 7.57%, P<0.001) and ‘emergent’, have pre-operative ventilator dependence within 48 hours of surgery (26.29% vs 15.59%, P<0.001), and septic shock (8.76% vs 4.01%, P<0.05) (Table 1).

TABLE 1
Comparisons of Baseline Patient Demographics and Pre-operative
Characteristics Between Alive and Deceased Patients. Data are
presented as number and percentage (n, %), ASA indicates American
Society of Anesthesiologists, age categories in years

| | Alive | | Deceased | | Total | | |
|---|---|---|---|---|---|---|---|
| | (n = 1,097) | | (n = 194) | | (n = 1,291) | | P-value |
| Gender | | | | | | | 0.248 |
| Male | 731 | (66.64) | 121 | (62.37) | 852 | (65.99) | |
| Age Categories | | | | | | | 0.049 |
| Age 20-30 | 5 | (0.46) | 1 | (0.52) | 6 | (0.46) | |
| Age 31-40 | 6 | (0.55) | 3 | (1.55) | 9 | (0.70) | |
| Age 41-50 | 22 | (2.01) | 6 | (3.09) | 28 | (2.17) | |
| Age 51-60 | 90 | (8.20) | 27 | (13.92) | 117 | (9.06) | |
| Age 61-70 | 259 | (23.61) | 52 | (26.80) | 311 | (24.09) | |
| Age 71-80 | 398 | (36.28) | 64 | (32.99) | 462 | (35.79) | |
| Age 81-90 | 287 | (26.16) | 38 | (19.59) | 325 | (25.17) | |
| Age 90+ | 30 | (2.73) | 3 | (1.55) | 33 | (2.56) | |
| Body Mass Index | | | | | | | 0.029 |
| Normal | 378 | (34.46) | 46 | (23.71) | 424 | (32.84) | |
| Underweight | 31 | (2.83) | 6 | (3.09) | 37 | (2.87) | |
| Overweight | 343 | (31.27) | 67 | (34.54) | 410 | (31.76) | |
| Obese | 345 | (31.45) | 75 | (38.66) | 420 | (32.53) | |
| Race | | | | | | | 0.044 |
| White | 990 | (90.25) | 162 | (83.51) | 1152 | (89.23) | |
| Asian | 29 | (2.64) | 10 | (5.15) | 39 | (3.02) | |
| African American | 70 | (6.38) | 20 | (10.31) | 90 | (6.97) | |
| Other | 8 | (0.73) | 2 | (1.03) | 10 | (0.77) | |
| Ethnicity | | | | | | | 0.052 |
| Non-Hispanic | 1008 | (91.89) | 186 | (95.88) | 1194 | (92.49) | |
| Hispanic | 89 | (8.11) | 8 | (4.12) | 97 | (7.51) | |
| Transfer Status | | | | | | | 0.006 |
| Home | 570 | (51.96) | 108 | (55.67) | 678 | (52.52) | |
| Acute Care Hospital | 346 | (31.54) | 43 | (22.16) | 389 | (30.13) | |
| Nursing Home | 33 | (3.01) | 3 | (1.55) | 36 | (2.79) | |
| Other | 27 | (2.46) | 4 | (2.06) | 31 | (2.40) | |
| Outside Emergency | 121 | (11.03) | 36 | (18.56) | 157 | (12.16) | |
| Department | | | | | | | |
| ASA Classification | | | | | | | <0.001 |
| III | 17 | (1.55) | 3 | (1.55) | 20 | (1.55) | |
| IV | 997 | (90.88) | 152 | (78.35) | 1149 | (89.00) | |
| V | 83 | (7.57) | 39 | (20.10) | 122 | (9.45) | |
| Emergency Case | 345 | (31.45) | 103 | (53.09) | 448 | (34.70) | <0.001 |
| Wound | | | | | | | 0.769 |
| Classification | | | | | | | |
| Clean | 1023 | (93.25) | 177 | (91.24) | 1200 | (92.95) | |
| Clean/Contaminated | 17 | (1.55) | 4 | (2.06) | 21 | (1.63) | |
| Contaminated | 7 | (0.64) | 2 | (1.03) | 9 | (0.70) | |
| Dirty/Infected | 50 | (4.56) | 11 | (5.67) | 61 | (4.73) | |
| Functional Health | | | | | | | 0.078 |
| Prior to Surgery | | | | | | | |
| Independent | 970 | (88.42) | 182 | (93.81) | 1152 | (89.23) | |
| Partially Dependent | 102 | (9.30) | 9 | (4.64) | 111 | (8.60) | |
| Totally Dependent | 25 | (2.28) | 3 | (1.55) | 28 | (2.17) | |
| Pre-operative | 171 | (15.59) | 51 | (26.29) | 222 | (17.20) | <0.001 |
| Ventilator | | | | | | | |
| Dependence | | | | | | | |
| Diabetes | | | | | | | 0.626 |
| No | 750 | (68.37) | 138 | (71.13) | 888 | (68.78) | |
| Non-Insulin Dependent | 146 | (13.31) | 26 | (13.40) | 172 | (13.32) | |
| Insulin Dependent | 201 | (18.32) | 30 | (15.46) | 231 | (17.89) | |
| Current Smoker | 219 | (19.96) | 31 | (15.98) | 250 | (19.36) | 0.195 |
| Dyspnea | | | | | | | 0.024 |
| No | 528 | (48.13) | 109 | (56.19) | 637 | (49.34) | |
| With Moderate Exertion | 424 | (38.65) | 55 | (28.35) | 479 | (37.10) | |
| At Rest | 145 | (13.22) | 30 | (15.46) | 175 | (13.56) | |
| Systemic Sepsis | | | | | | | 0.011 |
| within 48 Hours | | | | | | | |
| of Surgery | | | | | | | |
| None | 833 | (75.93) | 130 | (67.01) | 963 | (74.59) | |
| System Inflammatory | 183 | (16.68) | 40 | (20.62) | 223 | (17.27) | |
| Response | | | | | | | |
| Sepsis | 37 | (3.37) | 7 | (3.61) | 44 | (3.41) | |
| Septic Shock | 44 | (4.01) | 17 | (8.76) | 61 | (4.73) | |

Logistic Regression, Machine Learning, and GBM Modeling: Thirty-eight pre-operative variables were accounted for in the ACS-NSQIP calculator, including 16 patient co-morbidity related factors, 12 laboratory values, and 10 factors related to patient demographics and disposition were included in multi-variable logistic regression with a combination of forward selection and backward elimination. After stepwise selection, twelve relevant predictive factors remained for further analysis. Logistic regression modeling demonstrated acceptable accuracy (0.848; 95% Confidence Interval [95% CI] 0.808-0.882) and area under the curve (AUC 0.624; 95% CI 0.548-0.699).

Following variable selection, an automated machine learning approach returned the top five models with respect to AUC (Table 2). Gradient boosted machine followed by various Stacked Ensemble models demonstrated the best model performance. Gradient boosted machine modeling was used for further investigation as this was the top prediction model.

TABLE 2
Auto-machine learning models area under the
curve (AUC) results. Stacked ensemble models
with individual models used in parenthesis.

| Machine Learning Model | Area Under the Curve |
|---|---|
| Gradient Boosting Machine | 0.637 |
| Stacked Ensemble 1 | 0.63 |
| Stacked Ensemble 2 | 0.628 |
| Stacked Ensemble 3 | 0.627 |
| Stacked Ensemble 4 | 0.623 |

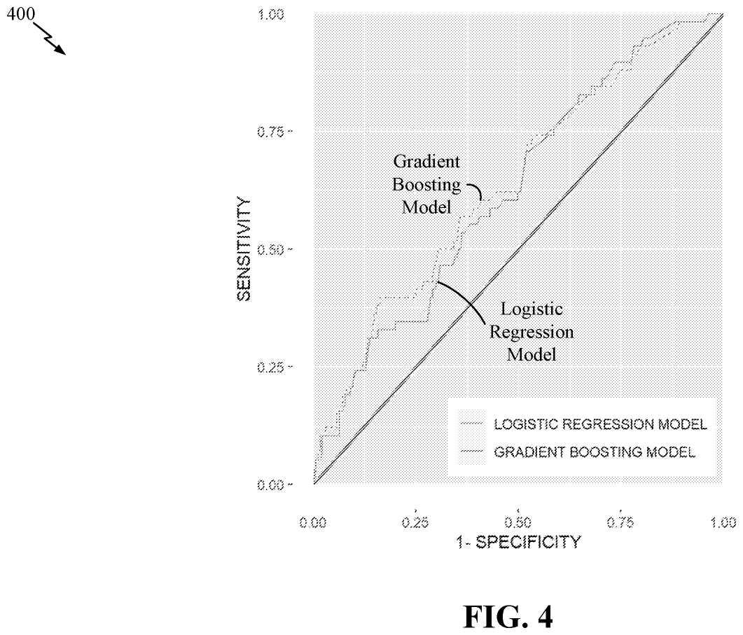

Gradient boosting machine modeling demonstrated the top five contributors to mortality for the overall cohort with acceptable performance (model accuracy 0.8501; 95% CI 0.811-0.884, AUC 0.637; 95% CI 0.559-0.715) (FIG. 4). The top five variables' relative influence on GBM modeling included (% relative influence on model prediction): cases classified as emergent (18.76%), elevated pre-operative alkaline phosphatase (15.42%), ventilator dependence within 48 hours of surgery (10.06%), body mass index (BMI) classified as overweight (8.36%) and obesity (8.07%).

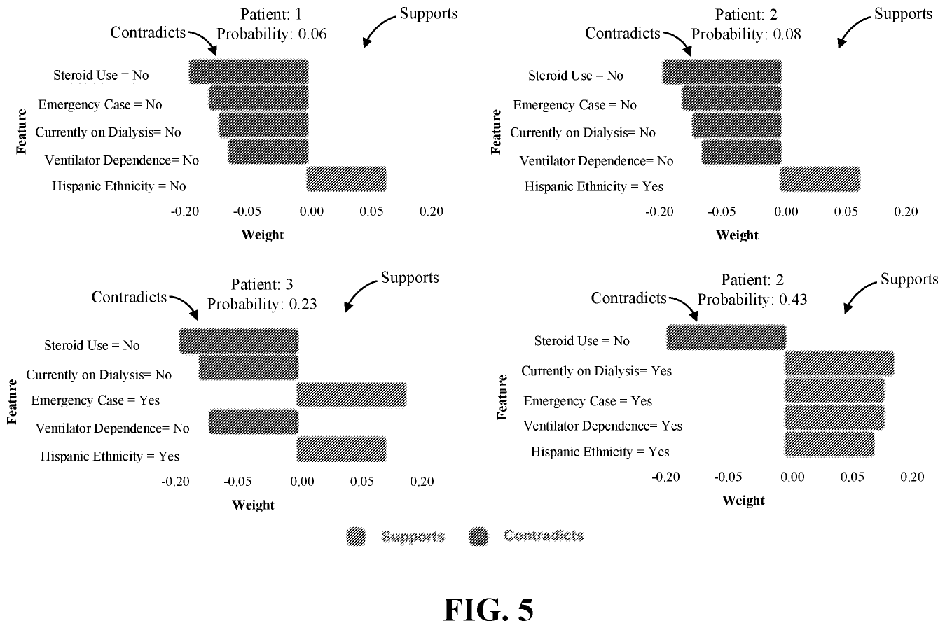

Local Interpretable Model-Agnostic Explanations Modeling: A local interpretable model-agnostic explanations approach was implemented to determine patient specific contributors to mortality and elucidate the feature weight as it argues for or against patient survival. Individualized features and weights on survivability were returned for the entire cohort (FIG. 5). In FIG. 5, the probability of survival (probability) is shown by NSQIP calculator, feature values (y-axis) and feature weight's effect as they support or contradict survival (x-axis). Further, personalized predicted survival probability and model accuracy were displayed. Patients with features contributing to survival displayed positive feature weight, whereas the presence of factors that contributed to mortality exhibited negative feature weight of varying magnitude corresponding to their contribution to survivability.

Combination Confusion Matrices: To further explore the additive effect of the top five factors as they contributed to mortality, combination confusion matrices of all possible combinations were calculated in combinations of two factors or more (Table 3). Specificity for mortality prediction improved in an additive fashion corresponding with a satisfactory negative predictive value in the absence of factors. As no patient had all five factors in this cohort, results are reported for combinations up to four factors. Model accuracy and specificity demonstrated the following: two factor combinations 76.1% (specificity 0.845), three factor combinations 83.4% (specificity 0.981), four factor combinations 84.9% (specificity 0.998).

TABLE 3
Combination confusion matrices results of top four
factors related to mortality including all possible
permutations of each factor combination

| Number of | | | | Negative |
|---|---|---|---|---|
| Factors | Accuracy | | | Predictive |
| Present | (95% CI) | Sensitivity | Specificity | Value |
| 2 | 0.761 | 0.284 | 0.845 | 0.869 |
| | (0.736-0.784) | | | |
| 3 | 0.839 | 0.036 | 0.981 | 0.852 |
| | (0.818-0.859) | | | |
| 4 | 0.849 | 0.005 | 0.998 | 0.85 |
| | (0.828-0.868) | | | |

Patient counseling and clinical decision making in patients undergoing high-risk cardiac surgery can present unique challenges. While current risk models may provide predicted morbidity and mortality percentages, the context in which variables contribute to these predictions and the complex interplay between features is often unknown. Herein, we present a machine learning approach to high-risk cardiac surgery risk scoring using a sequential ensemble decision tree model and an innovatively applied local interpretable model-agnostic explanations technique that accurately predicts mortality and identifies the relative contribution of risk factors in individualized patients.

Prudent clinical decision making is desirable in ensuring optimal patient outcome, especially in those with pre-operative factors predisposing to an increased risk of mortality. Recognition and mitigation of existing risk factors that contribute to patient mortality prior to intervention may allow for improved patient outcome. Alternatively, the recognition of irreversible factors may assist in patient and family counseling when considering not proceeding with invasive procedures. Whereas existing risk calculators provide estimated probability of morbidity and mortality, this modeling provides individualized context to the top contributing factors on mortality and their relative contribution to predictive modeling. Utilization of this technique may uncover modifiable risk factors that allow for actionable intervention, resulting in a decreased risk of mortality. Univariate analysis demonstrated deceased patients were more likely overweight or obese, to present in transfer from an outside emergency department, ASA Classification Class V, and have pre-operative ventilator dependence or septic shock. Personalized results were returned for each patient in the cohort with individualized prediction of survivability and the top factors and their weights contributing to survival outcome (FIG. 5). The ability to optimize an individual patient's most contributory factors on mortality prior to surgical intervention may provide rationale for delaying surgical intervention when feasible to improve outcome, or indeed offer justification for abandoning invasive interventions altogether in cases of extreme risk without modifiable factors. Fundamental to continued progress in optimizing risk scoring strategies using large datasets are clinician's ability to understand and interpret results relevant for their patients and the ease of applicability to daily practice. In this study, a gradient boosting machines approach provided insight into variable contribution to mortality. The contributory features on model performance, including cases classified as ‘emergent’, ventilator dependence, and obesity, provide important insights into factors associated with worse outcome. Additionally, LIME furnished individualized results for patients in this cohort as their factors argued for or against survivability. When considering the absence of the top factors contributing to mortality, combination confusion matrices demonstrated stepwise improvement in model specificity and accuracy (Table 3).

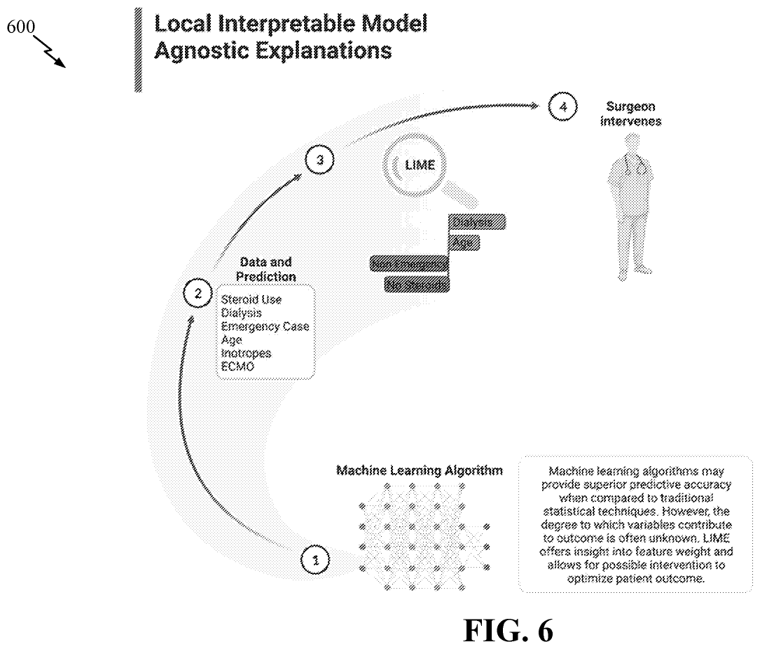

The application of machine learning algorithms to large clinical datasets has resulted in improved predictive accuracy as compared to traditional linear approaches in some instances. However the manner by which predictions are made using these approaches is often unclear. Consequently, a clinician's ability to “trust” predictions made using novel techniques may suffer and result in more accurate models remaining unused. The implementation of LIME allows for interpretability of a model's predictions which provides insight into individualized feature weight contribution to outcome (FIG. 6). FIG. 6 illustrates an example approach to interpretable machine learning using a local interpretable model agnostic explanations (LIME) approach. Machine learning algorithms may provide features contributing to mortality, though without model interpretability. By employing LIME, surgeons may better understand modifiable factors to mitigate operative mortality. Through the modification of a single data sample by alteration of the feature value and observation of the resultant impact on the measured outcome, LIME “interprets” predictions from each data sample. Additionally, to understand which portions of the interpretable model's inputs are contributing to prediction, LIME acts in a model-agnostic manner (i.e., LIME does not ‘peak’ into the model) by perturbing the input around its local neighborhood and observes how model predictions behave. The resultant series of interpretations characterize the contribution of each measured feature to a prediction for an individual patient. In this way, the user gains understanding of not only which features contribute to mortality, but the degree to which they contribute to the outcome. The employment of LIME in this context may encourage those unfamiliar with machine learning techniques to more readily adopt its use.

As machine learning applications are increasingly applied to large granular clinical datasets, the potential benefits of moving beyond traditional linear approaches in outcome prediction are being realized. There are several limitations of current existing technology should be considered. Modeling is inherently constrained to the number of variables and patients available in the NSQIP dataset and thus may be subject to selection bias. Moreover, because certain features of interest in the cardiac surgical population are not represented in the NSQIP database (i.e., pre-operative laboratory values, other existing co-morbidities, echocardiography, and heart catheterization data), generalizability to this cohort may be limited. Further validation of this analysis by replication in the STS ACSD database may be useful to determine applicability to this patient population. Additionally, outcomes such as 30-day hospital readmission, ICU length of stay, and hospital costs were not analyzed in this study. Evaluation and corroboration of findings in a trial setting is necessary to determine if mitigating identified factors in this analysis impacts patient outcome. Notwithstanding these limitations, this analysis and methodology provides rationale for further investigation of machine learning applications in the cardiac surgery cohort.

The application of a machine learning approach described in this disclosure to high-risk cardiac surgery using GBM and LIME offers individualized predicted mortality and identification of significant features and influence on mortality. Further exploration of this technique may be useful to inform operative decision making and family counseling in cases of substantial surgical risk.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

What is claimed is:

1. A system for patient-specific post-surgery mortality prediction, comprising:
a memory; and
a processor communicatively coupled to the memory;
wherein the memory stores a set of instructions which, when executed by the processor, cause the processor to:
receive a plurality of pre-operative factor indications for a patient;
obtain a first trained machine learning model and an interpretable model;
apply the plurality of pre-operative factor indications to the first trained machine learning model to obtain a plurality of confidence values corresponding to the plurality of pre-operative factor indications;
apply the plurality of confidence values to the interpretable model to obtain a plurality of interpretation indications, the plurality of interpretation indications corresponding to a subset of the plurality of pre-operative factor indications, the plurality of interpretation indications most contributing to mortality of the patient, the plurality of interpretation indications being specific to the patient; and
output a survival probability of the patient based on the plurality of interpretation indications.

2. The system of claim 1, wherein the plurality of pre-operative factor indications is at least one selected from the group of: patient co-morbidity related factor indications, laboratory test result indications, patient demographics and disposition related factor indications.

3. The system of claim 1, wherein the set of instructions, when executed by the processor, further cause the processor:
perform a combination of forward selection and a backward elimination to produce the plurality of pre-operative factor indications by reducing pre-operative factor dimensions.

4. The system of claim 1, wherein the first trained machine learning model comprises a gradient boost machine model.

5. The system of claim 1, wherein the interpretable model comprises a local interpretable model-agnostic explanation model.

6. The system of claim 5, wherein the local interpretable model-agnostic explanation model produces the plurality of interpretation indications by:
altering a first pre-operative factor indication of the plurality of pre-operative factor indications;
monitoring a resultant impact of the first pre-operative factor indication to the plurality of confidence values; and
producing the plurality of interpretation indications based on the resultant impact of the first pre-operative factor indication.

7. The system of claim 6, wherein a first interpretation indication of the plurality of interpretation indications corresponding to the first pre-operative factor indication among the subset comprises the first pre-operative factor indication and a weight of the first pre-operative factor indication, the weight being determined based on the resultant impact of the first pre-operative factor indication.

8. The system of claim 1, wherein the interpretable model produces each of the subset of the plurality of pre-operative factor indications and a respective weight of each of the subset of the plurality of pre-operative factor indications on the survival probability of the patient.

9. A system for patient-specific post-surgery mortality prediction model training, comprising:
a memory; and
a processor communicatively coupled to the memory;
wherein the memory stores a set of instructions which, when executed by the processor, cause the processor to:
receive a plurality of training datasets corresponding to a plurality of patients, each of the plurality of training datasets comprising: a plurality of pre-operative factor indications;
receive a plurality of ground truth datasets corresponding the plurality of patients, each ground truth dataset comprising a subset of the plurality of pre-operative factor indications; and
train a first machine learning model based on the plurality of training datasets and the plurality of ground truth datasets to obtain a plurality sets of confidence values, the plurality sets corresponding to the plurality of patients.

10. The system of claim 9, wherein the first trained machine learning model comprises a gradient boost machine model.

11. The system of claim 9, wherein the plurality of pre-operative factor indications is at least one selected from the group of: patient co-morbidity related factor indications, laboratory test result indications, patient demographics and disposition related factor indications.

12. The system of claim 9, wherein the set of instructions, when executed by the processor, further cause the processor:
perform a combination of forward selection and a backward elimination to produce the plurality of pre-operative factor indications by reducing pre-operative factor dimensions.

13. A method for patient-specific post-surgery mortality prediction, comprising:
receiving a plurality of pre-operative factor indications for a patient;
obtaining a first trained machine learning model and an interpretable model;
applying the plurality of pre-operative factor indications to the first trained machine learning model to obtain a plurality of confidence values corresponding to the plurality of pre-operative factor indications;
applying the plurality of confidence values to the interpretable model to obtain a plurality of interpretation indications, the plurality of interpretation indications corresponding to a subset of the plurality of pre-operative factor indications, the plurality of interpretation indications most contributing to mortality of the patient, the plurality of interpretation indications being specific to the patient; and
outputting a survival probability of the patient based on the plurality of interpretation indications.

14. The method of claim 13, wherein the plurality of pre-operative factor indications is at least one selected from the group of: patient co-morbidity related factor indications, laboratory test result indications, patient demographics and disposition related factor indications.

15. The method of claim 13, wherein the set of instructions, when executed by the processor, further cause the processor:
perform a combination of forward selection and a backward elimination to produce the plurality of pre-operative factor indications by reducing pre-operative factor dimensions.

16. The method of claim 13, wherein the first trained machine learning model comprises a gradient boost machine model.

17. The method of claim 13, wherein the interpretable model comprises a local interpretable model-agnostic explanation model.

18. The method of claim 17, wherein the local interpretable model-agnostic explanation model produces the plurality of interpretation indications by:
altering a first pre-operative factor indication of the plurality of pre-operative factor indications;
monitoring a resultant impact of the first pre-operative factor indication; and
producing the plurality of interpretation indications based on the resultant impact of the first pre-operative factor indication, the plurality of interpretation indications being indicative of contribution of the first pre-operative factor indication to a prediction for the patient.

19. The method of claim 18, wherein a first interpretation indication of the plurality of interpretation indications corresponding to the first pre-operative factor indication among the subset comprises the first pre-operative factor indication and a weight of the first pre-operative factor indication, the weight being determined based on the resultant impact of the first pre-operative factor indication.

20. The method of claim 13, wherein the interpretable model produces each of the subset of the plurality of pre-operative factor indications and a respective weight of each of the subset of the plurality of pre-operative factor indications on the survival probability of the patient.
